(19) United States
(12) Patent Application Publication
(10) Pub. No.: US 2013/0150375 A1
(43) Pub. Date: Jun. 13, 2013

(54) GEPIRONE-ER TREATMENT OF MAJOR DEPRESSION IN PATIENTS TAKING NON-STEROIDAL ANTI-INFLAMMATORY DRUGS (NSAIDs)

(71) Applicant: Fabre-Kramer Pharmaceuticals, Inc., Houston, TX (US)

(72) Inventors: Louis F. FABRE, Houston, TX (US); Stephen J. Kramer, Houston, TX (US)

(73) Assignee: Fabre-Kramer Pharmaceuticals.Inc., Houston, TX (US)

(21) Appl. No.: 13/709,986

(22) Filed: Dec. 10, 2012

Related U.S. Application Data
(60) Provisional application No. 61/568,962, filed on Dec. 9, 2011.

(51) Int. Cl. A61K 31/506; A61K 45/06

(52) U.S. Cl. CPC A61K 31/506; A61K 45/06
USPC 514/252.18

(57) ABSTRACT

A method of treatment of depression in a person also taking non-Steroidal anti-inflammatory drugs (NSAIDs) by administering gepirone as either a short term or long term therapy.

### FIELD OF THE INVENTION

The present invention relates to a method of treatment of depression in a person also taking Non-Steroidal Anti-inflammatory Drugs (NSAIDs) by administering gepirone as either a short term or long term therapy. More specifically, the present invention relates to a method of treating depression in mammals by administering to a subject in need thereof an effective amount of the high-dosage extended release formulation of gepirone in accordance with the present invention.

### BACKGROUND OF THE INVENTION

Gepirone (also known as 4,4-dimethyl-1-[4-[-(2-pyrimidinyl)-1-piperazinyl]-butyl]-2,6-piperidinedione hydrochloride) can be obtained by the process described in Example 7 of Temple, U.S. Pat. No. 4,423,049 (which is incorporated herein in its entirety by reference) and has the following structure:

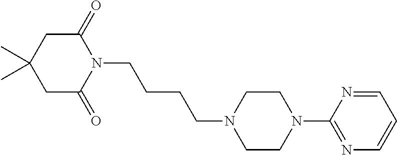 [SMILES: CC1(C)CC(=O)N(CCCCN2CCN(c3ncccn3)CC2)C(=O)C1]

Depressive disorders including major depressive disorder (MDD) are among the most common psychiatric conditions and affect as many as one in five individuals (1). According to recent studies such as “sequenced treatment alternatives to relieve depression (STAR-D) (2), as many as one-third of patients with depression are non-responsive to antidepressant medications, in particular, the selective serotonin reuptake inhibitors (SSRIs) and serotonin nor-epinephrine reuptake inhibitors (SNRIs) (3).

A “cytokine hypothesis” of depression has been advanced with claims that depressed persons have abnormal levels of certain cytokines. A model has been put forth which claims that SSRI efficacy in alleviating depression is mediated by an effect on cytokines (4). Widely used NSAIDs (aspirin, ibuprofen, indomethacin, naproxen, celecoxib, piroxicam and others) are claimed to have an opposing action on cytokines and to diminish or inhibitor the antidepressant action of SSRIs. Therefore, depressed persons who routinely take NSAIDs for concurrent medical conditions such as headache or arthritis do not respond to antidepressant drugs such as SSRIs.

This theory has been supported by an analysis of 1546 human subjects with major depression enrolled in the STAR-D study for which concomitant medication records were available (5). Only one measure of the effectiveness of antidepressant medication was used—the number (%) of treated subjects attaining remission at 12 weeks. Remission was defined as 5 or less on the QIDS-C16, a measure of depressive symptoms. 628 of 1137 depressed subjects (55%) attained remission in those that did not take NSAIDs. For those taking NSAIDs, only 182 of 409 (45%) achieved remission. These results are statistically significant p=0.0002. Therefore, it is claimed that the reason SSRI medications are ineffective in one-third of depressed patients is that many of them concomitantly take NSAIDs.

The antidepressant mechanism of action of SSRIs is complicated. Prior to an effect on cytokines, SSRIs have an effect on the receptor 5-HT_{1A} (6). Gepirone-ER an antidepressant with proven efficacy (7, 8) also has been shown to have a mechanism of efficacy including the receptor 5-HT_{1A}. Therefore, it would be expected that NSAIDs would interfere with the antidepressant effect of gepirone-ER in the same way as NSAIDs interfere with the antidepressant effect of SSRIs.

However, the present inventors have surprisingly found that there was no effect of NSAIDs on the efficacy of gepirone-ER at either dose level for any of the three efficacy measures. This finding shows that patents with depression can be successfully treated with gepirone-ER even if they are concomitantly taking NSAIDs.

### REFERENCE DOCUMENTS

(1) Kessler, R C, et al Lifetime prevalence and age-of-onset distributions of mental disorders in the World Health Organization's World Mental Health Survey Initiative. World Psychiatry 2007, 6:168-176
(2) Rush A J, et al Acute and longer term outcomes in depressed outpatients, requiring one or several treatment steps: A Star*D report. AM J Psychiatry 2006 163:1905-1917.
(3) Rush A J, Fava M, Wisniewski S R, et al.: Sequenced Treatment Alternatives to Relieve Depression (STAR*D): rationale and design. Control Clin Trials 2004, 25:119-142.
(4) Dunn A J, Swiergiel A H, de Beaurepaire. Cytokines as mediators of depression: What can we learn from animal studies? Neurosci Biobehav Rev 2005 29:891-909.
(5) Warner-Schmidt J L, Vanover K E, Chen E Y, Marshall J J, Greengard P. Antidepressant effects of selective serotonin reuptake inhibitors (SSRIs) are attenuated by anti-inflammatory drugs in mice and humans. Proc Natl Acad Sci USA 2011; 108:9262-9267.
(6) EFFICACY OF FLUOXETINE IN OUTPATIENTS WITH MAJOR DEPRESSION. Fabre, L. F., Jr. and Crismon, L., Current Therapeutic Research, 37(1):115-123, January, 1985
(7) Feiger A D, Heiser J F, Shrivastava R K, Weiss K J, Smith W T, Sitsen J M, Gibertini M. Gepirone extended-release: new evidence for efficacy in the treatment of major depressive disorder. J Clin Psychiatry 2003; 64:243-9.
(8) Bielski R J, Cunningham L, Horrigan J P, Londborg P D, Smith W T, Weiss K. Gepirone extended-release in the treatment of adult outpatients with major depressive disorder: a double-blind, randomized, placebo-controlled, parallel-group study. J Clin Psychiatry 2008; 69:571-7.

### SUMMARY OF THE INVENTION

It is an object of the present invention to provide a method of treating depression in a subject taking NSAIDs comprising administering a pharmaceutically effective amount of gepirone.

The pharmaceutically effective amount of gepirone may be contained in a pharmaceutical composition containing:
(1) from about 14 to about 25 wt % gepirone, or bioactive metabolite thereof, as a free base or a pharmaceutically acceptable salt thereof;
(2) from about 70 to about 85 wt % of a pharmaceutically acceptable cellulosic polymer matrix; and
(3) suitable amounts of one or more pharmaceutically acceptable excipients, wherein the release rate of gepirone from the dosage form is such that about 18 to 24 hrs are required to attain from about 90 to about 95% absorption of gepirone.

In one embodiment, component (3) further comprises at least one pharmaceutically acceptable excipient selected from the group consisting of a colorant, microcrystalline cellulose, colloidal silica and magnesium stearate; (2) is hydroxypropylmethylcellulose having a viscosity of from about 15,000 cps to about 100,000 cps; and (a) ranges from about 15.5 to about 18.7 wt %.

In another embodiment, the pharmaceutical composition contains:
(a) from about 14.0 to about 24.4 wt % gepirone, or bioactive metabolite thereof, as a free base or a pharmaceutically acceptable salt thereof,
(b) from 70.5 to 82.1 wt % hydroxypropylmethylcellulose having a viscosity of from about 15,000 to about 100,000 cps,
(c) 0 to about 1 wt % colorant
(d) About 8.0 to 16.7 wt % microcrystalline cellulose,
(e) about 0.39 to about 0.47 wt % colloidal silica, and
(f) about 0.29 to about 1.0 wt % magnesium stearate.

In another embodiment, the pharmaceutical composition contains:
(a) about 15.6 wt % of gepirone hydrochloride,
(b) about 75.3 wt % hydroxypropylmethylcellulose,
(c) about 0.31 wt % yellow ferric oxide,
(d) about 8.0 wt % microcrystalline cellulose, and
(e) about 0.42 wt % colloidal silica, and
(f) about 0.31 wt % magnesium stearate.

In another embodiment, the pharmaceutical composition contains:
(a) about 19.5 wt % of gepirone hydrochloride,
(b) about 70.7 wt % hydroxypropylmethylcellulose,
(c-1) about 0.24 wt % yellow ferric oxide,
(c-2) about 0.61 wt % red ferric oxide,
(d) about 8.2 wt microcrystalline cellulose,
(e) about 0.39 wt % colloidal silica, and
(f) about 0.29 wt % magnesium stearate.

In each of the above formulations the pharmaceutical composition may be in tablet form.

In a particular embodiment the tablet has an ovoid-rectangular shape with either biconvex or flat faces. In this object, the tablet has an overall dimension of 0.400±0.05 inches by 0.325±0.05 inches with a thickness of 0.240±0.025 inches (biconvex faces) or a thickness of 0.195±0.025 inches (flat faces). Also, within this object, the total weight of said tablet preferably ranges from 350 to 450 mg.

An object of this invention is to provide a method of ameliorating depression, anxiety, or psychological disorders (in particular depression) in mammals taking at least one non-steroidal anti-inflammatory drug (NSAID) by administering to a patient (in particular a human) in need thereof an effective amount of the composition or tablet form of the composition in accordance with the present invention. Additional aspects and embodiments of the invention may be found in the following detailed description.

### DETAILED DESCRIPTION OF THE INVENTION

Unless specifically defined, all technical and scientific terms used herein have the same meaning as commonly understood by a skilled artisan in biochemistry, chemistry, pharmacology, and the medical sciences.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

The present invention is based, in part, on the finding that patients with depression can be successfully treated with gepirone even if they are concomitantly taking NSAIDs. Previous studies with other anti-depressants have found negative effects on the antidepressive properties of other anti-depressant compounds when administered to patients who are taking NSAIDs. Accordingly, the result discovered by Applicants is surprising in view of the state of the art at the time of the present invention.

The gepirone used in the method of the present invention can be either extended release or immediate release.

Extended release drug forms (especially gepirone) offer several advantages over immediate release systems. Patient compliance is better because the extended release dosage forms need to be taken only one in a 24 hour period. Thus, plasma concentration levels do not vary unacceptably—i.e. give high initial drug levels that are associated with the incidence of unwanted side effects, as well as having rapid drops in drug levels to below therapeutic levels—when the ER dosage forms are administered. In addition, in the case of gepirone, due to the slow time release 1-PP levels are maintained at a satisfactory level thus avoiding significant complications arising from this metabolite.

In one possible embodiment, the present invention comprising administering a pharmaceutical composition for making an oral extended release gepirone dosage form comprising:
(1) from about 14 to about 25 wt % gepirone, or bioactive metabolite thereof, as a free base or a pharmaceutically acceptable salt thereof;
(2) from about 70 to about 85 wt % of a pharmaceutically acceptable cellulosic polymer matrix; and
(3) suitable amounts of one or more pharmaceutically acceptable excipients, wherein the release rate of gepirone from the dosage from is such that about 18 to 24 hours are required to attain from about 90 to about 95% absorption of gepirone.

In another possible embodiment, the present invention comprises administration of an extended release oral dosage form of gepirone, containing, in percentages expressed by weight:
(a) about 14.0 to about 24.4% (preferably, about 15.5 to about 18.7%) gepirone, or bioactive metabolite thereof, as a free base or a pharmaceutically acceptable salt thereof, for example a hydrochloride salt,
(b) about 70.5 to about 82.1% hydroxypropylmethylcellulose having a viscosity of from about 15,000 to about 100,000 cps.,
(c) 0 to about 1% (preferably 0 to about 0.3%) iron oxide,
(d) about 8.0 to about 16.7% microcrystalline cellulose,
(e) about 0.39 to about 0.47% (preferably about 0.42 to about 0.47%) colloidal silica, and
(f) about 0.29 to about 1.0% magnesium stearate.

Although the above embodiments are defined in terms of particular compounds for each component, it is to be understood that within the scope of the present invention these components may be replaced individually or in various combinations as described below. For each of the components below, it is to be understood that the recited weight percentage reflects the total concentration of each component. For example, if there is a mixture of compounds that fall within the scope of a defined component group the recited weight percentage reflects the total for that mixture of compounds.

Component (a)

In a particular embodiment, component (a) is present in the extended-release oral dosage form in a weight percentage of about 14.0 to about 24.4%, more preferably about 15.5. to about 18.7%. It is particularly preferred that component (a) be gepirone or a pharmaceutically acceptable salt thereof.

In vivo, gepirone is metabolized resulting in to major pharmaceutically active metabolites: 1-(2-pyrimidinyl)piperazine (1-PP) and 3′-OH gepirone (D. S. Robinson et al, Clinical Therapeutics, pp. 1618-1633 (2003)). Two additional metabolites that may also possess bioactivity are also formed in vivo: 5-OH gepirone and 3′,5-dihydroxy gepirone.

In humans, the release of 1-PP, a common azapirone metabolite, is believed to be responsible for adverse side effects, including dizziness, nausea, headache, and drowsiness. Moreover, 1-PP is a presynaptic α-2-adrenoceptor antagonist and it has been reported that it did not exhibit antidepressant-like characteristics in pre-clinical tests (D. S. Robinson et al, Clinical Therapeutics, pp. 1618-1633 (2003)). In contrast, the bioactive metabolites (3′-OH gepirone, in particular) have significant affinity for 5-HT receptors. 3′-OH gepirone has been demonstrated to modify 5-HT neurotransmission in a comparable manner to gepirone even though 3′-OH has been found to exhibit full agonism at post-synaptic receptors in the hippocampus, whereas gepirone is a partial agonist (D. S. Robinson et al, Clinical Therapeutics, pp. 1618-1633 J2003)).

In view of the foregoing, in an embodiment of the present invention one or more of the bioactive gepirone metabolites 3′-OH gepirone, 5-OH gepirone, and 3′,5-dihydroxy gepirone, or a pharmaceutically acceptable salt thereof, may be used in place of gepirone.

Further, although the pharmaceutically acceptable salt form is preferred, gepirone or the bioactive gepirone metabolites may be in a hydrate form, an enantiomeric form or mixture, or crystal form.

The pharmaceutical compounds suitable for administration in the present invention may be hydrochloride salts, but the free bases and other pharmaceutically acceptable salts are also suitable. The term “pharmaceutically acceptable salt” is well known in the art and the artisan is directed to S. M. Berge, (J. Pharmaceutical Sciences, 66: 1-9 (1997), incorporated herein by reference) for a further description. Suitable pharmaceutically acceptable salts for administration in the present invention include acid addition salts. The acid addition salt may be formed by mixing a solution of the compounds with a solution of a pharmaceutically acceptable non-toxic acid such as hydrochloric acid, hydrobromic acid, fumaric acid, maleic acid, succinic acid, acetic acid, citric acid, tartaric acid, carbonic acid, phosphoric acid, perchloric acid, sulphuric acid, oxalic acid, or malonic acid. Where the compound carried an acidic group, for example a carboxylic acid group, the present invention also contemplates salts thereof, preferably non-toxic pharmaceutically acceptably salts thereof, such as sodium, potassium, and calcium salts thereof.

Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethane-sulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pictate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, salts of amine groups. Salts of amine groups may also comprise the qua-ternary ammonium salts in which the amino nitrogen atom carries an alkyl, alkenyl, alkynyl or aralkyl group, nontoxic ammonium, quaternary ammonium, and amine cations formed using counterions such as halide, hydroxide, carboxylate, sulfate, phosphate, nitrate, lower alkyl sulfonate, and aryl sulfonate.

Component (b):

In a preferred embodiment, component (b) is present in the extended-release oral dosage form in a weight percentage of about 70.5 to about 82.1%. Preferably, component (b) is hydroxypropylmethylcellulose having a viscosity of from about 15,000 to about 100,000 cps, which is a release-controlling polymer & binder (a.k.a., sustaining agent).

For oral formulations and dosage forms, the use of a polymeric cellulose matrix, or sustaining agent, is preferred. Suitable matrixes include hydroxyalkylsubstituted alkylcelluloses having viscosities of about 15,000 cps to about 100,000 cps. Examples of acceptable hydroxymethyl propylcellulose (HPMC) samples include grades KI5M and KIOOM (i.e., 15,000 and 100,000 cps, respectively).

HPMC may be replaced (all or in part) or added to in the present invention, the replacement of some or the entire HPMC matrix may be with dicalcium phosphate or lactose, each of which generally increases dissolution rates.

In addition to the binder of component (b), the formulation of the present invention may also contain auxiliary binding agents, such as syrup, acacia, gelatin, sorbitol, tragacanth, or polyvinylpyrrolidone. Examples of suitable fillers or extenders also include starches (e.g., maize-starch), lactose, sorbitol, glycine, sucrose, glucose, mannitol, and silicic acid, sodium citrate and dicalcium phosphate. Examples of other suitable binders may include chitosan, alginates, gelatin, polyvinylpyrrolidinone, sucrose, acacia, and mixtures thereof.

In an embodiment of the present invention the ratio of gepirone to binder (gepirone:binder) ranges from 1:3.5 to 1:14.5. Preferably, the binder is hydroxymethylpropylcellulose (HPMC).

Component (c):

In a preferred embodiment, component (c) is present in the extended-release oral dosage form in a weight percentage of 0 to about 1%, preferably 0 to about 0.30. Although component (c) may be any colorant, it is preferred that the colorant be an iron oxide. In a particularly preferred embodiment the iron oxide is red ferric oxide, yellow ferric oxide, or mixtures thereof.

Additional exemplary colorants that may be used in place of or in addition to the foregoing include, but are not limited to, FD&C and D&C lakes, titanium dioxide, iron oxides, natural pigments, or dyes approved for ingestion by the U.S. Federal Drug Administration, or combinations thereof.

Component (d):

In a preferred embodiment, component (d) is present in the extended-release oral dosage form in a weight percentage of about 8.0 to about 16.70. Although component (d) may be any diluent and/or compression aid, it is preferred that the diluent/compression aid be microcrystal-line cellulose.

Additional diluents may include, but are not limited to, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

In yet another embodiment of the present invention, the ratio of gepirone to diluent (gepirone:diluent) ranges from 1:3 to 2.5:1. Preferably, the diluent is microcrystalline cellulose.

Component (e):

In a preferred embodiment, component (e) is present in the extended-release oral dosage form in a weight percentage of about 0.39 to about 0.470, preferably about 0.42 to about 0.470. Although component (e) may be any glidant, it is preferred that the glidant be colloidal silica (colloidal silicon dioxide).

Additional glidants include, but are not limited to, cornstarch, talc, or stearic acid, or combinations thereof.

Component (f):

In a preferred embodiment, component (f) is present in the extended-release oral dosage form in a weight percentage of about 0.29 to about 1.0. Although component (f) may be any lubricant, it is preferred that the lubricant be magnesium stearate.

Additional lubricants may include, but are not limited to cellulose, talc, polyethylene glycol, silicas, sodium lauryl sulfate, calcium stearate, and mixtures thereof.

Alternative Components:

It is contemplated in the present invention that the extended-release oral dosage form may also contain, in addition to components (a) through (f), various additional components. Based on the description above and Remington's Pharmaceuticals Sciences, 18th Edition (incorporated herein by reference), especially Part 8 therein, “Pharmaceutical Preparations and Their Manufacture, it would be well within the purview of the skilled artisan to add new compounds to the preferred embodiments described above.

In an embodiment of the present invention, the extended-release oral dosage form may include one or more additional pharmaceutically acceptable carriers. As used herein, the term “pharmaceutically acceptable carrier” means a non-toxic, inert solid, semi-solid or liquid filer, diluent, encapsulating material or formulation auxiliary. Some examples of materials which can serve as pharmaceutically acceptable carriers are already mentioned above, therefore, it is understood that in this aspect of the invention, pharmaceutically acceptable carriers may be any type of filer, diluent, encapsulating material or formulation auxiliary not previously mentioned.

In another embodiment, the extended-release oral dosage form may include may also include one or more releasing agents, coating agents, sweetening, flavoring and perfuming agents, preservatives and antioxidants.

The flavorant(s), which is used primarily for taste- and/or odor-masking, may be vanillin, sodium citrate, citric acid, mint, orange, lemon oil, or any other pharmaceutically approved flavorant or tastemasking agent, and combinations thereof.

Shape and Physical State of the High-Dosage Extended-Release Form of Gepirone

The present invention contemplates extended-release oral dosage forms that are suitable for orally administration. These forms include tablets, capsules, caplets, lozenges, powders, suspensions, syrups and the like are suitable forms. Preferentially, the tablet may be mentioned including those having a convex shape, a spherical (i.e., round) shape, or a capsule shape.

In a preferred embodiment, the extended-release oral dosage form of gepirone is a tablet. Preferably, the tablet has oval shape, which enhances their surface area and improves the release of gepirone therefrom. More preferably, the tablet has an ovoid-rectangular shape, which may have either flat or biconvex faces.

When the extended-release oral dosage form of gepirone is a tablet having an ovoid-rectangular shape, it is preferred that the tablets have an overall dimension of 0.400±0.05 inches by 0.325±0.05 inches with a thickness of 0.240±0.025 inches (biconvex faces) or 0.195±0.025 inches (flat faces).

In another embodiment of the present invention the overall tablet weight ranges from 350 to 450 mg, preferably from 375 to 425 mg, more preferably from 385 to 410 mg. Further, within the context of the present invention, the tablet weight range of 350 to 450 mg, preferably from 375 to 425 mg, more preferably from 385 to 410 mg correspond to the total weight of the unit dose form.

Therefore, the pharmaceutical compositions (i.e., extended-release oral dosage form of gepirone) of the present invention preferably contain between about 55 to 100 mg (preferably from 60 to 80 mg, more preferably 60 or 80 mg) of the active ingredient per unit dose.

The tablets may, if desired, be coated using known methods and excipients that may include enteric coating using for example hydroxypropylmethylcellulose phthalate. Such tablets may, if desired, be provided with enteric coatings by known methods, for example by the use of cellulose acetate phthalate. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Similarly, capsules, for example hard or soft gelatin capsules, containing the active compound with or without added excipients, may be prepared by known methods and, if desired, provided with enteric coatings in a known manner. The contents of the capsule may be formulated using known methods so as to give sustained release of the active compound. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Solid compositions of a similar type may also be employed as fillers in soft and hard-filled gelatin capsules using such excipients as lactose or milk sugar as well as high molecular weight polyethylene glycols and the like.

### Treatment Methods:

Gepirone can be orally administered in once-a-day extended release dosage forms, which contain gepirone hydrochloride (or a bioactive metabolite thereof), a cellulosic polymer matrix and suitable amounts of pharmaceutical excipients. The resultant gepirone formulation yields oral products the take about 19 to about 24 hours to release 90 to 95% of the active agent.

Therefore, in yet another embodiment of the present invention is a method of treating a patient diagnosed as suffering from major (primary or general) depression, with or without generalized anxiety disorder, comprising administering to said patient an effective amount of the extended-release oral dosage form gepirone as described in the present application.

In this embodiment, the patient (preferably a human, but inclusive of all mammals) in need of treatment is first diagnosed as suffering from a particular depressive condition. For a discussion of patient evaluation, diagnostic criteria, etc. of anxiety and depression, the skilled artisan is referred to: DSM-IV-TR (Diagnostic and Statistical Manual of Mental Disorders, American Psychiatric Association, Washington, D.C., 2000; Depressive Disorders: 369-428, and Anxiety Disorders: 429-484).

To this end, the gepirone extended-release composition and dosage forms of the invention are designed to deliver an effective anti-depressive and/or anxiolytic amount of gepirone or a pharmaceutically acceptable salt thereof to a mammal, preferably a human patient.

“Effective” doses as used herein refer to an amount of about 0.01 to 40 mg/kg body weight/day are contemplated, preferably 0.05 to 20 mg/kg body weight/day, more preferably 0.1 to 2 mg/kg of body weight/day. For certain central nervous system disorders, 15 to 90 mg/day, preferably 30-60 mg/day, are recommended. See U.S. Pat. No. 4,771,053 and U.S. Pat. No. 5,478,572. Alternatively, the effective dose or delivery system should result in plasma concentrations in the range of about 1 ng/ml to about 20 ng/ml, preferably about 1 ng/ml to about 5 ng/ml.

As described above, in an embodiment of the present invention, the extended-release oral dosage form of gepirone for use in the inventive method contains, expressed in weight percent:

(a) about 14.0 to about 24.4% (preferably, about 15.5 to about 18.7%) gepirone, or bioactive metabolite thereof, as a free base or a pharmaceutically acceptable salt thereof, for example a hydrochloride salt,

(b) about 70.5 to about 82.1% hydroxypropyl-methylcellulose having a viscosity of from about 15,000 to about 100,000 cps,

(c) 0 to about 1% (preferably 0 to about 0.3%) iron oxide,

(d) about 8.0 to about 16.7% microcrystalline cellulose,

(e) about 0.39 to about 0.47% (preferably about 0.42 to about 0.47) colloidal silica, and

(f) about 0.29 to about 1.0% magnesium stearate.

As stated herein above, gepirone in the extended-release oral dosage form of gepirone also embraces the bioactive gepirone metabolites selected from the group consisting of 3′-OH gepirone, 3′,5-dihydroxy gepirone, and 5-OH gepirone. The method may employ anyone of these compounds. However, combinations of these metabolites, or combinations of the metabolites with other active or inert ingredients, are also contemplated.

The present invention further provides methods for ameliorating depression, anxiety, or psychological disorders in a patient (preferably human, but inclusive of all mammals) in need of such treatment who is also taking NSAIDs, by administering to the patient an effective amount or dose of gepirone or a bioactive gepirone metabolite such as 3′-OH gepirone, 3′,5-dihydroxy gepirone, and 5-OH gepirone. As used herein, the administration of gepirone or a bioactive gepirone metabolite includes the administration of any active salt form, hydrate form, enantiomeric form or mixture, or crystal form of the compound.

Also within the scope of the present invention are methods in which the extended-release oral dosage form of gepirone is administered (sequentially or simultaneously) with one or more anti-depressive or anxiolytic agents in a combination therapy for treatment of depression and/or anxiety of patients who are taking at least one NSAID.

### NSAIDs

Depression and pain often occur at the same time. 65% of depressed patients suffer from one or more pain symptom, and from 5 to 85% of patients with pain will suffer from at least one form of depression (Bair M J, Robinson R L, Katon W. Depression and Pain Comorbidity: A Literature Review. Archives of Internal Medicine, 2003; 163 (20):2433-45).

Nonsteroidal Anti-inflammatory Drugs (NSAIDs) are used primarily to treat inflammation, mild to moderate pain, and fever. Specific uses can include but are not limited to treatment of headaches, arthritis, sports injuries, and menstrual cramps. Aspirin in particular can also be used to inhibit the clotting of blood and prevent strokes and heart attacks. As such, a significant number of people who suffer from depression are also at one point during treatment also consuming NSAIDs to treat said conditions.

NSAIDs work by blocking the COX enzymes that produce prostaglandins, which are a family of chemicals that promote inflammation, pain, fever, support the blood clotting function of platelets, and protect that lining of the stomach from the damaging effects of acid. NSAIDs block said COX enzymes reducing the prostaglandins in the body and in turn reduce inflammation, pain, fever, and other conditions not limited to those noted above.

Examples of NSAIDs which may be taken include but are not limited to the following: asprin, acetaminophen, celecoxib, diclofenac, diflunisal, etodolac, ibuprofen, indomethacin, ketoprofen, ketorolac, nabumetone, naproxen, oxaprozin, piroxicam, salsalate, sulindac, and tolmetin. These NSAIDS may be taken alone or in various combinations.

The depression referred to in the present application can include but is not limited to; major depressive disorder (also known as recurrent depressive disorder, clinical depression, major depression, unipolar depression, or unipolar disorder), melancholic depression, atypical depression, catatonic depression, postpartum depression, mental and behavioral disorders associated with the puerperium, and seasonal affective disorders.

### NSAID Dosage

The NSAID may be administered in capsule, powder, packet, tablet or any other palatable form. The NSAID may be administered at various frequencies, including weekly, twice a week, daily, twice a day, three times a day, four times a day, every 4 hours, and various times in between. The NSAID may be taken at the same time as the gepirone, at the same time plus at additional times, or at separate times. The dosage forms for each of the above listed NSAIDs, both alone and in combination, include but are not limited to anywhere from 75 mg to 2000 mg, including particular dosages ranging from 75 mg to 250 mg, 250 mg to 1500 mg, from 300 mg to 1200 mg, from 325 to 650 mg, from 500 mg to 1000 mg, and from 400 mg to 800 mg, which may be administered at any (or more) of the frequencies noted above.

The NSAID and the gepirone can be part of the same formulation. The NSAID and gepirone can be concurrently consumed, wherein concurrently consumed means that the NSAID and the Gepirone are consumed at the same time or within 1 hr of each other. In addition, the NSAID and gepirone may be taken separately at time intervals varying from 1 to 24 hrs.

### Production Methods:

The extended-release oral dosage form of gepirone of the present invention may be made by the following procedure: (i) admixing all or a part of components (a) through (f), (ii) blending the mixture, (iii) adding a part of or the remainder (if any) of components (a) through (f), (iv) blending the mixture (if remainder added during (iii)), (v) slugging, (vi) milling, (vii) adding the remainder of the lubricant (component (f); if part reserved during (i) and (iv)), (viii) blending the mixture; (ix) compressing the final finished tablet blend.

Of course it is to be understood that the aforementioned steps may be rearranged or substituted so long as the rearrangement or substitution does not substantially alter the pharmacological efficacy of the resultant dosage form from the process described above. In this regard, the artisan is referred to Remington's Pharmaceuticals Sciences, 18th Edition (incorporated herein by reference), for example Part 8 therein, “Pharmaceutical Preparations and Their Manufacture.

Further, the artisan is directed to the processing procedures described in U.S. Pat. Nos. 4,423,049 and 5,478,572 (incorporated herein by reference) as providing relevant examples of the extended-release oral dosage form of gepirone production process described above.

In a preferred embodiment, the following procedure is followed to make the extended-release oral dosage form of gepirone:

i) admixing all of component (a), all of component (c), all of component (e), and 20% of the total concentration of component (b);

ii) blending the mixture from (i);

iii) adding to the blend from (ii) all of component (d), half of the total concentration of component (f), and the remaining 80% of the total concentration of component (b);

iv) blending the mixture from (iii);

v) slugging the blend from (iv);

vi) milling the slug from (v);

vii) adding the remaining 50% of component (f) to the milled slug from (vi);

viii) blending the mixture from (vii);

ix) compressing the blend from (viii) into the final desired form.

Typically, the resultant blends are compressed in step (ix) into tablets or into micropellets. If micropellets are made, they are optionally overcoated with conventional coating adjuvant(s) and then tableted or filled into capsules.

In the case where an enteric coating is desired, this coating would typically be added following step (ix).

It is to be understood that each of the above-described manufacturing processes may be scaled-up to an industrially applicable scale and is in no way limited to individual dosage preparation methods. It is also contemplated that alterations to each step (individually or collectively) may be made to facilitate the production of the inventive extended-release oral-dosage form of gepirone, so long the modifications do not alter the composition of the final product beyond the tolerance limits described above or the bioefficacy of the resultant product.

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of,” “chosen from,” and the like include mixtures of the specified materials.

Where a numerical limit or range is stated herein, the endpoints are included. Also, all values and subranges within a numerical limit or range are specifically included as if explicitly written out. In this regard, where the term “about” is recited, this term is understood to include the recited value, as well as values that are within 1% of the recited value (either above or below depending upon whether “about” defines an upper or lower boundary), preferably with 0.50 of the recited value.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only, and are not intended to be limiting unless otherwise specified.

### EXAMPLES

Similar to the study of STAR-D (Reference (2)), a study was conducted to determine the antidepressant effect of gepirone-ER in studies involving 2398 depressed subjects. To corroborate previous work, a population of 312 depressed patients taking the SSRI fluoxetine was also studied. To control for a placebo effect, 1045 depressed patients taking placebo were studied. Finally, 1041 depressed subjects taking gepirone-ER were studied. In contrast to the STAR-D study, three measures of efficacy were employed: remitters (HAMD-17 score 7 or less at end of treatment), responders (HAMD-17 50% reduction from baseline score at end of treatment) and HAMD-17 total score (change from baseline at end of treatment). Further, to test a possible dose of NSAID effect, two dose levels were tested: subjects taking any NSAID any time and subjects taking daily aspirin and other NSAIDs.

The following Examples represent a sampling of the subjects studied:

### Example 1

A 60 y/o man came to a psychiatric clinic complaining of depression. His symptoms included: depressed mood, insomnia, lack of energy, low sex drive, and mild suicidal thoughts. He also was being treated for cardiac complains and was taking a daily NSAID (aspirin) to improve blood flow. He refused to discontinue his aspirin because his cardiologist had prohibited him from doing so. His initial HAMD-17 was 26. After 8 weeks of treatment with Gepirone-ER all of his depressive symptoms had been eliminated. His HAMD-17 was 0 and he was very much improved.

### Example 2

A 51 year old woman came into the same psychiatric clinic complaining of depression. Her symptoms included depressed mood, lethargy, guilt, and trouble waking up early in the morning. She was also being treated by her neurologist for chronic headaches with ibuprofen. She refused to discontinue the ibuprofen because the headaches interfered with her ability to work. Her initial HAMD-17 was 16. After 8 weeks of treatment with fluoxetine (an SSRI) she showed no improvement and her HAMD-17 was 20. A further trial of a second SSRI (citalopram) also failed.

The results (Table 1) indicate that the STAR-D results for SSRIs are confirmed. That is, NSAIDs interfere with the antidepressant activity of an SSRI (fluoxetine). At the high NSAID dose, the responder analysis reveals fluoxetine plus NSAIDs 45%; fluoxetine alone 55% (p=0.09), a trend toward statistical significance. Also for the high dose group, the HAMD-17 change from baseline was −9.04 for the fluoxetine plus NSAIDs, compared to −10.95 for the fluoxetine alone group, statistically significantly (p=0.02).

There was no effect of NSAIDs on the efficacy of gepirone-ER at either dose level for any of the three efficacy measures, as there was no effect on patients treated with placebo.

These results indicate that patients with depression can be successfully treated with gepirone-ER even if they are concomitantly taking NSAIDs.

TABLE 1
Effect of NSAIDs on Antidepressant Efficacy for SSRIs (Fluoxetine) and Gepirone-ER

| Population | Remitters¹ | P value² | Responders³ | P value⁴ | HAMD-17 CFB | P value⁵ |
|---|---|---|---|---|---|---|
| Fluoxetine (n-312) | | | | | — | |
| Any NSAID | 29% | | 48% | | −9.57 | |
| None | 28.3% | 0.96 | 51% | 0.50 | −10.5 | 0.23 |
| High NSAID | 27% | | 45% | | −9.03 | |
| None | 32% | 0.35 | 55% | 0.09 | −10.95 | 0.02 |
| Gepirone-ER (n = 1041) | | | | | | |
| Any NSAID | 23% | | 37% | | −8.58 | |
| None | 23% | 0.89 | 41% | 0.49 | −8.8 | 0.96 |
| High NSAID | 24% | | 36% | | −8.25 | |
| None | 25% | 0.67 | 40% | 0.24 | −8.87 | 0.24 |
| Placebo (n = 1045) | | | | | | |
| Any NSAID | 23% | | 36% | | −8.33 | |
| None | 24% | 0.67 | 36% | 0.97 | −8.0 | 0.86 |
| High NSAID | 26% | | 40% | | −8.4 | |
| None | 24% | 0.40 | 35% | 0.13 | −8.1 | 0.24 |

¹A remitter means a depressed patient who after treatment has a HAMD-17 score of 7 or less (i.e. no applicable depressive symptoms after treatment).
²The first P value is “% remitters.” This is calculated on the percentages by an odds ratio program.
³A responder is a patient with 50% improvement in HAMD-17.
⁴The second P value is for “% responders” and is calculated the same way.
⁵The third P value is for HAMD-17 change from baseline (CFB) calculated by a one way analysis of variance.

1. A method of treating depression in a subject in need thereof, comprising administering to said patient an effective amount of gepirone, wherein said subject is taking at least one NSAID.

2. The method of claim 1, wherein said subject is human.

3. The method of claim 1, wherein said effective amount ranges from 0.01 to 40 mg/kg body weight per day.

4. The method of claim 1, wherein the gepirone is in the form of a pharmaceutical composition comprising: (a) from about 14 to about 25 wt % gepirone, or bioactive metabolite thereof, as a free base or a pharmaceutically acceptable salt thereof; (b) from about 70 to about 85 wt % of a pharmaceutically acceptable cellulosic polymer matrix; and (c) suitable amounts of one or more pharmaceutically acceptable excipients, wherein the release rate of gepirone from the dosage form is such that about 18 to 24 hours are required to attain from about 90 to about 95% absorption of gepirone.

5. The method of claim 4, wherein (c) further comprises at least one pharmaceutically acceptable excipient selected from the group consisting of colorant, microcrystalline cellulose, colloidal silica and magnesium stearate.

6. The method of claim 4, wherein the concentration of (a) ranges from about 15.5 to about 18.7 wt %.

7. The method of claim 4, wherein (a) is gepirone or a pharmaceutically acceptable salt thereof.

8. The method of claim 4, wherein (a) is a bioactive metabolite of gepirone or a pharmaceutically acceptable salt thereof.

9. The method of claim 8, wherein said bioactive metabolite of gepirone is selected from the group consisting of 3′-OH gepirone, 3′,5-dihydroxy gepirone, and 5-OH gepirone.

10. The method of claim 4, wherein said composition is in a form of a tablet.

11. The method of claim 10, wherein the total weight of said tablet ranges from 350 to 450 mg.

12. The method of claim 10, wherein the weight of (a) in said table ranges from 60 to 80 mg.

13. The method of claim 1, wherein the gepirone is in the form of a pharmaceutical composition comprising: (a) from about 14.0 to about 24.4 wt % gepirone, or bioactive metabolite thereof, as a free base or a pharmaceutically acceptable salt thereof, (b) from 70.5 to 82.1 wt % hydroxypropylmethylcellulose having a viscosity of from about 15,000 to about 100,000 cps., (c) 0 to about 1 wt % colorant, (d) about 8.0 to about 16.7 wt % microcrystalline cellulose, (e) about 0.39 to about 0.47 wt % colloidal silica, and (f) about 0.29 to about 1.0 wt % magnesium stearate.

14. The method of claim 13, wherein (a) ranges from about 15.5 to about 18.7 wt %.

15. The method of claim 1, wherein the NSAID is at least one selected from the group consisting of asprin, acetaminophen, celecoxib, diclofenac, diflunisal, etodolac, ibuprofen, indomethacin, ketoprofen, ketorolac, nabumetone, naproxen, oxaprozin, piroxicam, salsalate, sulindac, and tolmetin.

16. The method according to claim 1, wherein the NSAID is at least one selected from the group consisting of aspirin, acetaminophen and ibuprofen.

17. The method according to claim 1, wherein the depression is selected from the group consisting of major depressive disorder, melancholic depression, atypical depression, catatonic depression, postpartum depression, mental and behavioral disorders associated with the puerperium, and seasonal affective disorder.

18. The method according to claim 1, wherein the depression is major depressive disorder.

19. A pharmaceutical composition, comprising gepirone and an NSAID.

20. A method of treating depression in a subject in need thereof, comprising administering to said patient an effective amount of each gepirone and an NSAID.
